(19) United States
(12) Patent Application Publication
(10) Pub. No.: US 2023/0391716 A1
(43) Pub. Date: Dec. 7, 2023

(54) METHOD AND SYSTEM FOR PREPARING UREA BY COUPLING DENITRATION WITH ELECTROCATALYTIC REDUCTION

(71) Applicant: Tsinghua University, Beijing (CN)

(72) Inventors: Yue PENG, Beijing (CN); Junhua LI, Beijing (CN); Zhen CHEN, Beijing (CN); Haibo YIN, Beijing (CN); Rong WANG, Beijing (CN)

(21) Appl. No.: 18/035,625

(22) PCT Filed: Jan. 7, 2022
(86) PCT No.: PCT/CN2022/070761

§ 371 Date: May 5, 2023

(30) Foreign Application Priority Data
Jan. 22, 2021 (CN) 202110089811.3

(51) Int. Cl. C07C 273/16; B01D 53/78; B01D 53/56

(52) U.S. Cl. CPC C07C 273/16; B01D 53/78; B01D 53/56; B01D 2258/0283

(57) ABSTRACT

The present disclosure relates to a method and a system for preparing urea by coupling denitration with electrocatalytic reduction. Specifically, the method of the present disclosure comprises: introducing an absorption solution containing nitrite and nitrate, which is generated by subjecting flue gas containing nitric oxide to a denitration step, into a separation step to separate the nitrite and the nitrate, thereby obtaining a solution rich in the nitrite and the nitrate; and introducing the solution rich in the nitrite and the nitrate generated in the separation step into an electrocatalysis step, and carrying out the electrocatalytic reduction in the presence of carbon dioxide or carbonate ions to generate a urea solution. The system of the present disclosure comprises: a denitration device, a separation device connected to the denitration device, and an electrocatalytic device connected to the separation device. The method and system of the present disclosure can be used for the deep removal of nitrogen oxides from industrial flue gas at a low temperature, and can achieve the recycling of nitrogen.

### TECHNICAL FIELD

The present disclosure belongs to the field of environmental protection, and relates to a method and a system for preparing urea by coupling denitration with electrocatalytic reduction, in particular to a method and a device for purifying nitrogen oxides in industrial flue gas and recycling nitrogen.

### BACKGROUND

Nitrogen oxides generated from combustion of fossil fuels in industrial boilers and from various medium- and high-temperature processes are major air pollutants and major precursor pollutants to the formation of photochemical smog and haze, threatening human life and health and living environment. Therefore, deep control of nitrogen oxides emissions is an urgent need for environmental protection, and is of great significance to the ecological progress and sustainable development.

At present, for nitrogen oxides generated from medium and high temperatures processes of 300° C. to 400° C., the Selective Catalytic Reduction (SCR) technology is generally adopted with ammonia or urea as a reductant. Due to the restriction of temperature window of denitration catalysts, it is difficult to achieve the desirable denitration efficiency when the flue gas temperature is low. Accordingly, medium- and low-temperature flue gas is mainly treated by activated carbon desulfurization and denitrification integrated technology. However, the activated carbon is relatively expensive and suffers from serious abrasion during operation, so the overall operating cost is high. For medium-temperature flue gas below 100° C., it is still difficult to achieve satisfactory denitration efficiency even in the case of adopting the activated carbon adsorption technology.

The oxidation-absorption denitration technology can remedy the deficiencies in the above-mentioned technologies effectively. First, the NO in the flue gas that is poorly soluble in water is oxidized by homogeneous oxidation or heterogeneous catalytic oxidation to high-valence nitrogen oxides, such as N₂O₅, which have a relatively high solubility; and further the nitrogen oxides are absorbed by an alkaline absorption solution to realize flue gas purification. This technology has been applied in the flue gas treatment process of various industrial boilers, and shows a good denitration effect on nitrogen oxides in medium- and low-temperature flue gas. Nevertheless, the treatment of the nitrate solution resulting from the oxidation-absorption denitration technology with existing water treatment method is costly, and it is difficult to realize resource utilization of nitrogen effectively.

On the condition that the solution contains carbonate ions, it is possible to realize catalytic reduction of nitrate ions by means of electrocatalysis to generate urea, and the urea is further concentrated to form a urea solution at a certain concentration, which can be further purified and can be used as fertilizers or used in the SCR denitration process. This provides a very promising method for the treatment of nitrate in the oxidation-absorption denitration technology. However, there still remains blank about how to combine the oxidation-absorption denitration technology with the electrocatalytic reduction technology to form a technological route of denitrating industrial flue gas and recycling nitrogen.

Patent Literature 1 discloses a device and a method for diverting ozone oxidation coordinated absorbing flue gas desulfurization and denitration. In this patent, a diverting three-way joint is utilized to divide flue gas into two gas branches, and ozone is fed into only one of the gas branches, so as to realize efficient utilization of ozone by regulating proportion of the diverting three-way joint and the ozone feeding volume and effectively reduce the cost for oxidation. However, this patent does not mention how to treat waste water containing nitrites and nitrates.

Patent Literature 2 discloses a flue gas desulfurization and denitration system and method based on ozone synergistic catalytic oxidation. Through the synergistic effect of ozone and plasma, the oxidation efficiency of NO is increased, and urea is used for further treatment to enable the reaction of nitrites and urea to form nitrogen, carbon dioxide, and water. However, because the mass transfer coefficient of NO₂ is still very low, it is difficult to achieve the desirable absorption efficiency and guarantee the denitration efficiency in the wet absorption process.

Patent Literature 3 discloses a novel denitration system for efficiently removing nitrates from a water body by electrocatalytic hydrogen evolution and catalytic hydrogenation and use thereof, which belongs to the technical field of water treatment. This invention patent provides a technical solution concerning electrocatalytic reduction of nitrate ions in a water and the main target products are ammonia and nitrogen, which has the advantage of transportation safety compared with the hydrogen production by electrocatalytic reduction. However, this technology does not consider how to be combined with the removal process of nitrogen oxides in flue gas.

### CITATION LIST

Patent Literature 1: CN110052142B
Patent Literature 2: CN110876885A
Patent Literature 3: CN111533220A

### SUMMARY

### Technical Problem

In view of the above problems, an objective of the present disclosure is to provide a technology combined oxidation-absorption denitration with electrocatalytic reduction, and specifically, an objective of the present disclosure is to provide a method and a system for preparing urea by coupling denitration with electrocatalytic reduction.

### Solution to Problem

After painstaking researches, the inventors have found that the above-mentioned technical problem can be addressed by the following embodiments:

1. A method for preparing urea by coupling denitration with electrocatalytic reduction, wherein the method comprises:
introducing an absorption solution containing nitrite and nitrate, which is generated by subjecting flue gas containing nitric oxide to a denitration step, into a separation step to separate the nitrite and the nitrate, thereby obtaining a solution rich in the nitrite and the nitrate; and
introducing the solution rich in the nitrite and the nitrate generated in the separation step into an electrocatalysis step, and carrying out the electrocatalytic reduction in the presence of carbon dioxide or carbonate ions to generate a urea solution.

2. The method according to item 1 described above, wherein the denitration step comprises an oxidation step of oxidizing the nitric oxide in the flue gas to a high-valence nitrogen oxides, and an absorption and purification step of absorbing the high-valence nitrogen oxides with an alkaline absorption solution to generate the absorption solution containing the nitrite and the nitrate.

3. The method according to item 2 described above, wherein in the absorption and purification step, the alkaline absorption solution is one or more of solutions of calcium hydroxide, magnesium hydroxide, sodium hydroxide, and potassium hydroxide.

4. The method according to item 2 or 3 described above, wherein the absorption and purification step comprises an enhanced gas-liquid mass transfer step.

5. The method according to any one of items 1 to 4 described above, wherein in the separation step, one membrane material selected from an ion exchange membrane, a nanofiltration membrane, and a reverse osmosis membrane is used for the separation.

6. The method according to any one of items 2 to 5 described above, wherein the alkaline absorption solution having been subjected to the separation step is introduced into a fresh alkaline absorption solution and is reused in the absorption and purification step.

7. The method according to any one of items 1 to 6 described above, wherein the method further comprises a concentration step of heating the urea solution to concentrate urea.

8. A system for preparing urea by coupling denitration with electrocatalytic reduction, wherein the system comprises:
a denitration device, a separation device connected to the denitration device, and an electrocatalytic device connected to the separation device.

9. The system according to item 8 described above, wherein the system further comprises a concentration device connected to the electrocatalytic device.

10. The system according to item 8 or 9 described above, wherein the denitration device comprises an oxidation device and an absorption and purification device, and optionally, the absorption and purification device comprises an enhanced gas-liquid mass transfer unit.

### Advantageous Effects of Invention

By implementing the above technical solutions, the present disclosure can achieve the following technical effects:
(1) the present disclosure can not only achieve deep denitration of industrial flue gas containing nitrogen oxides, but also realize recycling of nitrogen by combining the oxidation-absorption denitration with the electrocatalytic reduction;
(2) the present disclosure improves the process of absorbing nitrogen oxides by homogeneously oxidizing or heterogeneously oxidizing the poorly soluble low-valence nitric oxide and combining an alkaline absorbent with the enhanced gas-liquid mass transfer technology to enhance the absorption effect on the nitrogen oxides, and the present disclosure can realize stable denitration.
(3) in the presence of carbon dioxide or carbonate ions, the separated nitrite and nitrate at a high concentration are allowed to generate urea by electrocatalytic reduction, and the urea is further concentrated and purified to form a practical urea product which is useful as a chemical fertilizer or a reductant for SCR denitration;
(4) the separated nitrite and nitrate at a low concentration may be introduced into the alkaline absorption solution to realize recycling; and
(5) the method according to the present disclosure enables consumption of carbon dioxide.
(6) In addition, the present disclosure may also take advantage of the waste heat in flue gas to generate electricity, supplying electric energy for the electrocatalytic reaction of the present disclosure.

### BRIEF DESCRIPTION OF THE DRAWINGS

FIG. 1: a schematic diagram of the purification system according to the present disclosure.

### DESCRIPTION OF REFERENCE NUMBERS

1: flue gas inlet; 2: oxidation device; 3: flue; 4: slurry pool; 5: enhanced gas-liquid mass transfer unit; 6: spray layer; 7: demister; 8: spray absorption tower; 9: purified gas outlet; 10: absorption solution storage tank; 11: membrane separation device; 12: carbon dioxide supplementary port; 13: electrocatalytic device; 14: vapor outlet; 15: evaporation and concentration device; 16: urea solution outlet; 17: fresh absorption solution supplementary port; 18: pump; 19: membrane module.

### DETAILED DESCRIPTION

The embodiments of the present disclosure will be described below, but the present disclosure is not limited thereto. The present disclosure is not limited to the configurations described below. Various alternations may be made within the scope of the invention. Embodiments and examples obtained by appropriately combining the technical means disclosed in different embodiments and examples are also encompassed in the technical scope of the invention. In addition, all of the literatures described herein are incorporated herein as references.

Unless otherwise defined, the technical and scientific terms used herein have the same meanings as typically understood by one of ordinary skill in the art to which the present disclosure belongs.

In the present specification, the numerical range represented by “numerical value A to numerical value B” or “numerical value A-numerical value B” refers to the range including the endpoint values A and B.

In the present specification, the term “may” means both that an operation is performed and that an operation is not perform. In the present specification, the term “optional” or “optionally” means that the event or situation described subsequently may or may not occur, and the description includes the case where the event occurs and the case where the event does not occur.

In the present specification, the phrases such as “some specific/preferred embodiments”, “other specific/preferred embodiments”, “some specific/preferred technical solutions”, and “other specific/preferred technical solutions” mean that particular elements (e.g., features, structures, properties and/or characteristics) described in relation to this embodiment are included in at least one of the embodiments described herein and may or may not be present in other embodiments. In addition, it should be appreciated that the elements may be combined in any appropriate manner in various embodiments.

The term “comprising” and any variation thereof used in the specification and claims as well as the appended drawing of the present disclosure is intended to cover non-exclusive inclusion. For example, a process, method or system, product or device comprising a series of steps or units is not limited to the listed steps or units, but optionally further comprises unlisted steps or units, or optionally further comprises other steps or units inherent to the process, method, product or device.

<The First Aspect>

According to the first aspect of the present disclosure, there is provided a method for preparing urea by coupling denitration with electrocatalytic reduction. By combining the oxidation-absorption denitration with the electrocatalytic reduction, the method of the present disclosure can not only achieve deep denitration of industrial flue gas containing nitrogen oxides, but also realize resource utilization of nitrogen.

The method for preparing urea from industrial flue gas by coupling oxidation-absorption denitration with electrocatalytic reduction according to the present disclosure may comprise the following steps: an oxidation step, an absorption and purification step, a separation step, an electrocatalysis step, and a concentration step.

The steps will be described in detail below.

Oxidation Step

In the oxidation step, the nitric oxide is oxidized by homogeneous oxidation or heterogeneous oxidation to high-valence nitrogen oxides in the flue.

The oxidation step of the present disclosure is not particularly limited, and oxidation methods commonly used in the art may be adopted. For example, an ozone oxidation method can be selected as the homogeneous oxidation method, and the ozone may be generated by high-voltage discharge; and a catalytic oxidation method can be selected as the heterogeneous oxidation method.

In a preferred embodiment of the present disclosure, the ozone oxidation method may be adopted. In this case, the ratio of ozone to NO may be 1 to 2, preferably 1 to 1.5, further preferably 1 to 1.2 on a molar ratio basis.

In some embodiments of the present disclosure, the catalytic oxidation method may be adopted. The catalyst may include a noble metal catalyst, an alloy catalyst, a metal oxide catalyst, etc. For example, examples of the catalyst may include Au, Ag, Pt, Pd, and alloys thereof, and TiO₂, V₂O₅, and perovskite-type composite oxides. The metal oxide catalyst is preferred from the standpoints of cost savings and catalysis efficiency.

In some embodiments of the present disclosure, the high-valence nitrogen oxides may be one or both of NO₂ and N₂O₅.

Absorption and Purification Step

In the absorption and purification step, the high-valence nitrogen oxides generated in the oxidation step are introduced into the spray tower where an alkaline absorption solution is used to absorb the nitrogen oxides in flue gas to generate an absorption solution containing nitrite and nitrate, thereby purifying the flue gas.

In the present disclosure, the alkaline absorption solution may be one or two or more of the solutions of calcium hydroxide, magnesium hydroxide, sodium hydroxide, and potassium hydroxide.

In a preferred embodiment of the present disclosure, the absorption and purification step may comprise an enhanced gas-liquid mass transfer step. The enhanced gas-liquid mass transfer step can improve the absorption effect on the nitrogen oxides to realize effective, stable and deep removal of nitrogen oxides.

In the enhanced gas-liquid mass transfer step of the present disclosure, the enhanced effect of mass transfer may be achieved by means of bubbling, etc.

Separation Step

In the separation step, a solution rich in nitrite and nitrate is separated from the absorption solution containing nitrite and nitrate generated in the absorption and purification step by membrane separation.

In the present disclosure, examples of the membrane material used for the membrane separation include an ion exchange membrane, a nanofiltration membrane, and a reverse osmosis membrane.

In a preferred embodiment of the present disclosure, the alkaline absorption solution having subjected to the separation step re-enters the absorption tower to recycle the absorption solution. The alkaline absorption solution having subjected to the separation step may contain a trace amount of nitrite and nitrate.

The solution rich in nitrite and nitrate generated in the separation step enters the electrocatalysis step.

Electrocatalysis Step

The solution rich in nitrite and nitrate generated in the separation step is introduced into the electrocatalysis step, and subjected to the electrocatalytic reduction in the presence of carbon dioxide or carbonate ions to produce a urea solution.

In the electrocatalysis step, the nitrite and nitrate are allowed to react with carbon dioxide or carbonate ions under the action of a catalyst and an external electric field to generate urea.

Since carbon dioxide may be utilized in this step, the step has a certain capability to consume carbon dioxide.

The catalyst used in this step is not particularly limited, and the catalysts commonly used in the art may be used. For example, the catalyst may be a metal catalyst or an alloy catalyst. Examples of the catalyst may include Ni, Co, Pt, Pd, Au, Ag, and alloys thereof.

Concentration Step

In the concentration step, the urea resulting from the electrocatalytic reduction is heated to concentrate the urea.

After the concentration step, a practical urea product may be produced. For example, it may be used as a chemical fertilizer or a reductant for SCR denitration, so as to realize resource utilization of nitrogen.

<The Second Aspect>

According to the second aspect of the present disclosure, there is provided a system for preparing urea by coupling denitration with electrocatalytic reduction. The system of the present disclosure integrates a denitration device with an electrocatalytic device, such that the nitrite and nitrate resulting from denitration are used directly to produce urea, thereby realizing resource utilization of nitrogen.

The system for preparing urea by coupling denitration with electrocatalytic reduction according to the present disclosure may include: a denitration device, a separation device connected to the denitration device, an electrocatalytic device connected to the separation device, and a concentration device connected to the electrocatalytic device. Of these, the denitration device includes an oxidation device and an absorption and purification device. The absorption and purification device may further include an enhanced gas-liquid mass transfer unit.

It is to be noted that the oxidation device, the absorption and purification device, the separation device, the electrocatalytic device, and the concentration device in the present disclosure may all use conventional devices in the art and are not particularly limited.

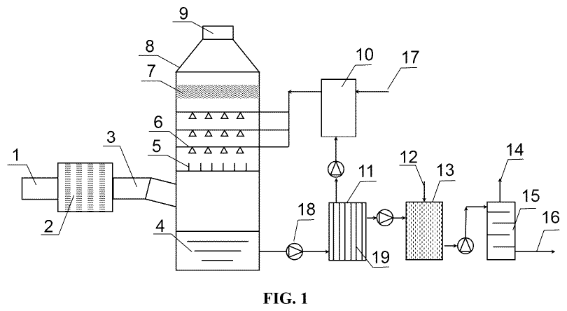

The devices will be described in detail below with reference to FIG. 1.

As shown in FIG. 1, the oxidation device 2 is disposed upstream of the spray absorption tower 8. In the oxidation device, NO is oxidized by ozone or in the presence of a catalyst to high-valence nitrogen oxides such as NO₂ and/or N₂O₅.

The spray absorption tower 8 includes a slurry pool 4, an enhanced gas-liquid mass transfer unit 5, a spray layer 6, a demister 7, and a purified flue gas outlet 9 therein. The nitrogen oxides entered the spray absorption tower 8 are absorbed by the alkaline absorption solution sprayed by the spray layer 6, and an absorption solution containing nitrate and nitrite is formed.

The slurry pool 4 is configured to accommodate the absorption solution containing nitrate and nitrite formed after absorption of the nitrogen oxides, and is connected to the membrane separation device 11, so that the absorption solution containing nitrate and nitrite enters the membrane separation device 11.

Furthermore, in the present invention, an enhanced gas-liquid mass transfer unit 5 may be provided, which is a member inside the spray tower that may form a bubble layer with a certain height, to enhance the efficiency of absorbing the nitrogen oxides and achieve deep purification.

Additionally, the spray tower of the present disclosure may be further provided with a demister 7 therein to remove the mist generated in the spray tower.

The membrane separation device 11 is provided with a membrane module 19 and provided with an absorption solution outlet (not shown in the FIGURE) and an outlet (not shown in the FIGURE) for the solution rich in nitrate and nitrite. The membrane module 19 may be one of an ion exchange membrane, a nanofiltration membrane, and a reverse osmosis membrane. The membrane module 19 may separate the nitrate and nitrite from the absorption solution to obtain a solution rich in nitrate and nitrite.

The absorption solution outlet of the membrane separation device 11 is connected to the absorption solution storage tank 10. The absorption solution storage tank 10 is connected to the spray layer 6 and is further provided with a fresh absorption solution supplementary port 17. Therefore, the alkaline absorption solution having been subjected to the separation device and a possible trace amount of nitrate and nitrite can be mixed with the fresh absorption solution and enter the spray layer 6, so that the absorption solution is reused to absorb the high-valence nitrogen oxides.

The outlet for the solution rich in nitrate and nitrite is connected to the electrocatalytic device 13. The electrocatalytic device 13 is provided with a carbon dioxide supplementary port 12. In the electrocatalytic device 13, the nitrate and nitrite are allowed to react with carbon dioxide or carbonate in the presence of a catalyst to form urea.

The electrocatalytic device 13 is connected to the evaporation and concentration device 15, so as to introduce the resulting urea into the evaporation and concentration device 15 for concentration and purification.

The evaporation and concentration device 15 is provided with a urea solution outlet 16 to export the concentrated urea product out of the system.

In the present disclosure, the electric energy consumed by the electrocatalytic device 13 may be low-grade electric energy including one or more of wind power, solar power generation, waste heat power generation, and power generation in excess of the grid-connected demand for the steady-state and hybrid operation of coal-fired units.

In addition, the spray absorption tower 8 and the membrane separation device 11, the membrane separation device 11 and the absorption solution storage tank 10, the membrane separation device 11 and the electrocatalytic device 13, and the electrocatalytic device 13 and the evaporation and concentration device 15 are connected via pumps 18 to realize media transport.

The denitration process and urea preparation process are detailed as follows:

As shown in FIG. 1, the industrial flue gas to be purified passes through the flue gas inlet 1 into the oxidation device 2. In the oxidation device 2, the NO which is poorly soluble in water can be oxidized by adding ozone or installing a catalyst to form high-valence nitrogen oxides such as NO₂ and N₂O₅. At this time, the oxidation step of NO may be regulated by adjusting the addition amount of ozone and the arrangement of the catalyst.

The oxidized nitrogen oxides pass through the flue 3 into the spray absorption tower 8, so that they are brought into contact with the alkaline absorption solution sprayed by the spray layer 6 to realize the absorption and purification of the nitrogen oxides. An enhanced gas-liquid mass transfer unit 5 may be arranged between the lowermost spray layer and the inlet of the spray absorption tower to form gas-liquid bubbling flow in a local area within the absorption tower, thereby enhancing the gas-liquid mass transfer effect and realizing deep purification of the nitrogen oxides. The purified flue gas is discharged from the purified gas outlet 9.

In the slurry pool 4, the absorption solution that absorbs the nitrogen oxides is enriched with the nitrate and nitrite at a certain concentration and the absorption solution is pumped into the membrane separation device 11 through the pump. The separation of the nitrate and nitrite is achieved by the membrane module 19 of the membrane separation device 11. The alkaline absorption solution having been subjected to the separation step and a possible trace amount of nitrate and nitrite enter the absorption solution storage tank 10 for re-entry into the spray absorption tower 8. The separated solution containing nitrate and nitrite at a higher concentration enters the electrocatalytic device 13.

Depending on the concentration of the nitrite ions and nitrate ions entering the electrocatalytic device 13, an appropriate amount of carbon dioxide may be added through the carbon dioxide supplementary port 12 or an appropriate amount of carbonate may be added to the electrocatalytic device 13, to form a mixed solution containing nitrite ions, nitrate ions, and carbonate ions in the electrocatalytic device 13. Under the action of a catalyst, urea is generated in the electrocatalytic device 13.

The resulting urea may enter the evaporation and concentration device 15 to be further concentrated and purified to form urea or a urea solution with an application value in the market. The resulting urea may be exported via the urea solution outlet 16. The other vapors are discharged through the vapor outlet 14.

The system for preparing urea by coupling denitration with electrocatalytic reduction according to the present disclosure has the advantages of compact structure, stable operation, and the capability to continuously treat large flow of industrial flue gas while achieving resource utilization of nitrogen.

### EXAMPLE

In order to enable a person skilled in the art to have a better understanding of the technical solution of the present disclosure, the technical solution of the present disclosure will be described in detail below with reference to a specific example.

### Example 1

In a cement kiln, with the adoption of the flue gas circulation process, the flue gas emission was 660000 m³/h, and the flue gas temperature was about 200° C.; the concentrations of the sulfur dioxide and nitric oxide in the flue gas were 800 mg/Nm 3 and 300 mg/Nm³, respectively. Ozone was used to oxidize the nitric oxide; a calcium hydroxide solution was used as an alkaline absorption solution; a nanofiltration membrane was used to filter the nitrate and nitrite; and a copper-palladium alloy was used as a catalyst for electrocatalytic reduction.

The detailed process was as follows:

The discharged flue gas first entered the oxidation device 2. Ozone was added at a molar ratio of O₃/NO=1.5 to oxidize NO to NO₂ and N₂O₅. The flue gas containing NO₂ and N₂O₅ entered the spray absorption tower 8 and was brought into contact with the calcium hydroxide solution as the alkaline absorption solution and then was absorbed by the absorption solution. In order to remove NO₂ with a relatively low absorption coefficient, the enhanced gas-liquid mass transfer unit 5 could be provided in the spray absorption tower 8 to form bubbling flow in a local area of the absorption tower and enhance the effect of the mass transfer, thereby realizing deep removal of the nitrogen oxides.

The absorption solution in the slurry pool 4 was rich in nitrate ions and nitrite ions. After the absorption solution entered the membrane separation device 11 composed of the nanofiltration membrane, the nitrate ions and nitrite ions were separated out and sent to the electrocatalytic device 13. At the same time, the alkaline absorption solution and a possible trace amount of nitrate ions and nitrite ions were sent into the absorption solution storage tank 10 and mixed with the fresh alkaline absorption solution, and then re-entered the spray absorption tower 8 through the spray layer 6.

In the electrocatalytic device 13, an appropriate amount of carbon dioxide was added through the carbon dioxide supplementary port 12 according to the amounts of the nitrate ions and nitrite ions. Under the action of the copper-palladium alloy as the catalyst, the nitrite(s) and nitrate(s) were reduced to urea. The resulting urea could enter the evaporation and concentration device 15 to be further concentrated and purified. In this example, the electric energy consumed by the electrocatalytic device 3 came from the waste heat of the flue gas.

### INDUSTRIAL APPLICABILITY

By coupling the oxidation-absorption denitration with the electrocatalytic reduction, the present disclosure can not only achieve deep denitration of the industrial flue gas containing nitrogen oxides, but also achieve recycling of nitrogen.

The foregoing is merely preferred embodiments of the present disclosure. It shall be appreciated that several improvements and variations may still occur to one of ordinary skill in the art without departing from the technical principle of the present disclosure, and these improvements and variations shall also be deemed to fall within the scope of protection for the present disclosure.

What is claimed is:

1. A method for preparing urea by coupling denitration with electrocatalytic reduction, wherein the method comprises:
introducing an absorption solution containing nitrite and nitrate, which is generated by subjecting flue gas containing nitric oxide to a denitration step, into a separation step to separate the nitrite and the nitrate, thereby obtaining a solution rich in the nitrite and the nitrate; and
introducing the solution rich in the nitrite and the nitrate generated in the separation step into an electrocatalysis step, and carrying out the electrocatalytic reduction in the presence of carbon dioxide or carbonate ions to generate a urea solution.

2. The method according to claim 1, wherein the denitration step comprises an oxidation step of oxidizing the nitric oxide in the flue gas to a high-valence nitrogen oxides, and an absorption and purification step of absorbing the high-valence nitrogen oxides with an alkaline absorption solution to generate the absorption solution containing the nitrite and the nitrate.

3. The method according to claim 2, wherein in the absorption and purification step, the alkaline absorption solution is one or more of solutions of calcium hydroxide, magnesium hydroxide, sodium hydroxide, and potassium hydroxide.

4. The method according to claim 2, wherein the absorption and purification step comprises an enhanced gas-liquid mass transfer step.

5. The method according to claim 1, wherein in the separation step, one membrane material selected from an ion exchange membrane, a nanofiltration membrane, and a reverse osmosis membrane is used for the separation.

6. The method according to claim 2, wherein the alkaline absorption solution having been subjected to the separation step is introduced into a fresh alkaline absorption solution and is reused in the absorption and purification step.

7. The method according to claim 1, wherein the method further comprises a concentration step of heating the urea solution to concentrate the urea.

8. A system for preparing urea by coupling denitration with electrocatalytic reduction, wherein the system comprises:
a denitration device, a separation device connected to the denitration device, and an electrocatalytic device connected to the separation device.

9. The system according to claim 8, wherein the system further comprises a concentration device connected to the electrocatalytic device.

10. The system according to claim 8, wherein the denitration device comprises an oxidation device and an absorption and purification device, and optionally, the absorption and purification device comprises an enhanced gas-liquid mass transfer unit.
